(19) United States
(12) Patent Application Publication
(10) Pub. No.: US 2018/0058618 A1
(43) Pub. Date: Mar. 1, 2018

(54) Tubing Connector for Decreased Contamination

(71) Applicant: Juan Nepomuc Walterspiel, Menlo Park, CA (US)

(72) Inventor: Juan Nepomuc Walterspiel, Menlo Park, CA (US)

(21) Appl. No.: 15/789,388

(22) Filed: Oct. 20, 2017

Related U.S. Application Data
(63) Continuation of application No. PCT/US2016/047058, filed on Aug. 15, 2016.
(60) Provisional application No. 62/180,201, filed on Jun. 16, 2015.

(51) Int. Cl. F16L 37/244; A61M 16/08; A61M 39/16; A61M 39/18

(52) U.S. Cl. CPC F16L 37/244; A61M 16/0875; A61M 39/165; A61M 2205/584; F16L 2201/44; F16L 2201/20; F16L 2201/10; A61M 39/18

(57) ABSTRACT

Embodiments of this disclosure include male and female connector parts for connecting elements of a fluid delivery device, the male and/or female elements having one or more resilient, flexible guards that can be moved laterally or compressed axially to avoid interference with coupling the male and female parts. Such connectors can be used to connect tubing, syringes, respirators, and other devices for fluid transport. Such connectors can decrease the likelihood of nosocomial infections.

### CLAIM OF PRIORITY

This Patent Cooperation Treaty (PCT) International patent application claims priority to U.S. provisional patent application No. 62/180,201, filed 16 Jun. 2015. This provisional application is incorporated herein fully by reference.

### FIELD OF THE DISCLOSURE

This disclosure relates generally to luer-type and other male to female and female to male connectors as well as temporarily sealed needleless access ports for use in delivering fluids to, taking fluids from, and draining fluid from subjects. Particularly, this disclosure relates to improved connectors having one or more protective ribs or guards and overlapping extensions that can be bent, compressed, and/or broken away for standard connector use, and any other type of connectors or couplings that are in use to connect tubes and syringes. This disclosure also relates to improved devices to delivering gases to, or taking bases from patients using ventilators, and to connectors used in biotechnology culture, or any other use in which sterility is desired.

### BACKGROUND

Standard connectors and other male to female and female to male connectors are used to connect tubes and syringes for the delivery of sterile fluids or gases, typically have a male element that is inserted into a female element with such couplings then reversibly secured by various screw-on and locking devices.

Human skin is colonized by a microbiome, or a mixture of colonizing microbes that typically includes coagulase negative staphylococci, which are the most common agents found to cause infections of indwelling catheter lines. Skin colonizing microorganisms can also comprise Staphylococcus aureus, a vast array of Gram-negative organisms, yeast, and fungi. Many of these microorganisms exhibit resistance to one or multiple antimicrobial agents, and have become very difficult to treat.

Similarly, connections between tubes or conduits connecting ventilators to patients are potential locations for contamination. Because ventilators often have water vapor that may condense on connectors, tubes, and conduits, bacterial and other undesirable organisms may colonize ventilator components, thereby causing increased morbidity and mortality.

Other uses include biotechnology, cell culture, and laboratory technologies in which maintaining a sterile conduit for fluids is desired.

Pathogenic organisms adhere easily to any protruding or otherwise exposed elements of standard intravenous lines and a variety of other male to female connectors as well as to needle and needleless injection ports. Accidentally caught microorganisms can grow into a biofilm, whose formation is aided by the fluid filled capillary spaces that are formed when a fluid coupling is made.

Free swimming (planktonic) organisms are released from such biofilms. They are carried into the bloodstream, colonize the inner surfaces of indwelling catheters and the clot(s) that invariably form at the end of infusion lines.

The colonization, clotting, and the inflammatory responses to these organisms cause catheter occlusions, emboli, signs and symptoms of inflammation, seeding into organs, and prompt diagnostic dilemmas, that lead to various diagnostic and therapeutic interventions, that apart from the avoidable human suffering caused, drive healthcare costs out of proportion to the small expense for the proposed passive protective devices.

### SUMMARY

In an embodiment, the sterile male element of a connector usually protrudes beyond a securing element (also known as “part” or “portion”) of the connector to ease the insertion into the female counterpart of a connector. This functional advantage is offset by the disadvantage of the protruding male or female element being exposed to accidental contact with non-sterile environmental surfaces that include the skin of a patient or the caregiver, furniture, clothing and the like. This risk of avoidable colonization and infection also pertains to all freely exposed parts of female insertion couplings also referred to as hubs, and the needle, as well as needleless injection ports that frequently have valves or membranes and are particularly prone to colonization with pathogens that subsequently cause line and catheter infections.

Because US healthcare providers are no longer being paid to treat nosocomial infections that could have been prevented, there is now a strong incentive for the modification of design of long-held and well-known connectors. This incentive also extends into the growing area of the more economic home health care and by necessity, home self-care.

The passive protective devices or guards of this disclosure address these economic and healthcare problems by reducing or preventing healthcare associated infections. Protective guards can include flexible elements or “ribs” that extend outwards from the connector element and also extend beyond the distal (open) end of a male or female lur connector element. When in use, these protective guards may make contact with a source of contamination, but keep the critical ends of the connectors from making contact with the source of contamination.

In certain embodiments, a guard can be made with flexible protective ribs or resilient fibers that can be moved from a relaxed position by forces used to connect complementary elements of a connector. In certain embodiments, a guard can be a solid element that can be fitted to a device. Even if a connection is not completed, and the connector elements must be re-engaged, the flexible protective guard can help maintain sterility of the device, and thereby decrease the likelihood of inadvertent contamination of the device and the patient. In certain embodiments, a guard is axially compressible to allow for swabbing of these connector elements.

### BRIEF DESCRIPTION OF THE DRAWINGS

This disclosure is described with reference to specific embodiments thereof. Other features can be appreciated with reference to the figures, in which:

FIG. 1 depicts a schematic drawing 100 of this disclosure having a longitudinal section (or cut through) of a male luer lock (bottom) and a protective guard element or rib (120a), made of a material that can easily bend. As shown, rib 120a can bend either upward 120b or downward 120c (dashed lines), or around the axis (not shown) from the male connector 112.

FIG. 2 depicts a schematic drawing 200 of this disclosure having bending ribs or guards of a shape that by a circular arrangement or assembly of multiple ribs 120 forms a receiving funnel or guard. The ribs point into their respective distal and proximal directions and are arranged around a typical luer lock type coupling extender that exhibits a male part below and a female needleless coupling above. Shown here in schematic form are also rotating rings 200a and 200b on which a rib assembly of multiple ribs can rotate, for the rib assembly or guard to easily fit into or bend out of the way of any protective provisions on a counter coupling.

FIG. 3 depicts a schematic drawing 300 of this disclosure having a protective rib 120a with one bending or breaking point 305.

FIG. 4 depicts a schematic drawing 400 of this disclosure having a protective rib 405 with repetitive variations in thickness or diameter along its length that create several bending or breaking points 410, 415, and 420.

FIG. 5A depicts a schematic drawing 500 of this disclosure having a protective rib 505a of undulant protective shape that easily would slide over or into impediments and bend away upon closing a connection when needed. 510 is a looped distal end of the rib 505a to facilitate sliding over protrusions.

FIG. 5B depicts a schematic drawing of this disclosure having a rib 505b and looped distal end 510 as shown in FIG. 5A, where rib is bent to increase coverage area to the side and flexibility.

FIG. 6 depicts a schematic drawing 600 of a guard of this disclosure having slanted rib 605 with a bending or breaking point 610, that in aggregate of two or more of such slanted ribs 605 form a funnel that would open and bend or break a protective rib apart when encountering an impediment upon the closing of a connection.

FIG. 7 shows a schematic drawing 700 of this disclosure having a perpendicular cross-sectional view cut through a rib assembly with one curved rib 120a protecting a male connector part 112, where the open element adds visibility for engagement and increased flexibility.

FIG. 8 depicts a schematic drawing 800 of this disclosure having protective rib 120a ending in a circular protecting ring 805 that would fit over or under an impeding part of a counter connecting coupling or protector.

FIG. 9 depicts a schematic drawing 900 of this disclosure having a rib 120a ending in two open circular protecting rings 905a and 905b that can fit over a counter connecting coupling or protector.

FIG. 10 depicts a schematic drawing 1000 of this disclosure having a protective circular guard (alternatively, a helical guard) 1005 that can be compressed out of the way or around of an impeding part during coupling. Such a circular guard can be used on a female connector element so a guard on a male connector element can be inserted inside the circular guard on a female connector part.

FIG. 11 depicts a schematic drawing 1100 of this disclosure having protective guard 1105 that comprises flexible, interlocking, connected and superimposed compressible spiral shaped ribs. Such a funnel shaped guard can be placed on a female connector element, and can be sized to permit a male connector element to fit within protective guard 1105.

FIG. 12 shows a schematic drawing 1200 of this disclosure having a protective rib assembly where ribs 120a are arranged on ring 1205 that can be rotated in a fitting groove on a connector. End loops 120d at the distal ends of ribs 120a to facilitate sliding of ribs 120a over protrusions.

FIG. 13 shows a schematic drawing 1300 of this disclosure having a protective guard where ribs 120a have loops 120d at their distal ends to facilitate sliding of ribs 120a over protrusions, and arranged on an open ring 1305 that can be slidably affixed to a connector part and rotated. This open ring can be clipped into a fitting groove on a connector.

FIG. 14A depicts a drawing 1400 of the prior art showing male luer connector having unprotected element 1405 and female luer needleless connector having unprotected element 1410.

FIG. 14B depicts “cage” guard 1407 (in circle) this disclosure over a male luer connector 1405 with openings 1412 on the sides. Such openings can increase visibility for proper alignment with a counterpart connector element. Slit 1413 can permit compression or expansion of cage guard 1407 to permit easier coupling to each other of the male and female connectors.

FIG. 14C depicts a drawing of female end 1410 of this disclosure having a needleless connector protected by a compressible circular protector 1411, where the compressibility of protector 1411 allows for swabbing and cleaning of exposed end 1410.

FIG. 14D depicts a drawing of a protected luer connector this disclosure having male end 1405 and female end 1410 connected together. Cage protector 1407 enclosing male end 1405 has incision 1413 on its lateral side that allows cage 1407 to have its diameter reduced or expanded when sliding into compressible protector 1411.

FIG. 15 depicts a drawing 1500 of a guard of this disclosure 1505 having four flap-like elements 1510.

### DETAILED DESCRIPTION

### Definitions

This disclosure includes some well-known terms and others that are defined below.

The term “connector” means a device used to connect tubing together.

The term “element” means a part of a device.

The term “connector part” or “luer part” means either a male or female element.

The term “luer” or “Luer” means a device for connecting two devices together to provide a central channel, through which a fluid or gas can flow between the devices. The term “luer” is not intended to be solely for use in intravenous lines, but also includes any connector where sterility of the interior of the device is desired. The term “luer” includes any connector used to couple fluid carrying device.

The term “male” means an element or part of a connector having a protruding end sized and adapted to fit with a counterpart “female” element.

The term “female” means an element or part of a connector having a receiving element sized and adapted to accommodate a “male” element.

The term “guard” or “protector” means a device affixed to an element that extends laterally, distally, or both laterally and distally beyond the end of an element to provide a shield to hinder a male or female element from making unintended contact with a source of potential contamination.

The term “fiber” or “rib” means a resilient element that alone or in combination with other fibers or ribs can form a guard.

The term “cage” means a configuration of a guard having a network of interdigitated or interlocking ribs, or a configuration is produced by molding resilient materials into a non-rigid but solid shape.

### DESCRIPTION OF EMBODIMENTS

Connectors of this disclosure include an outer addition to existing intravenous or gas line connectors and couplings. The new couplings of this disclosure provide passive prevention of avoidable healthcare associated infections, specifically of intravenous line, catheter infections that follow catheter hub colonization, or respirators. (1. DE 2843281 A1 Oct. 4, 1978 Apr. 10, 1980; 2. Linares J., Sitges-Serra A, Garau J., et al., Pathogenesis of catheter sepsis: a prospective study with quantitative and semiquantitative cultures of catheter hub and segments. J. Clin Microbiol 1985; 21:357; 3. Tenney J H Moody M R, Newman K A, et al., Adherent microorganisms on luminal surfaces of long-term intravenous catheters. Importance of Staphylococcus epidermidis in patients with cancer. Arch Intern Med 1986; 146:1949; 4. Miller J J, Venus B, Mathru M., Comparison of the sterility of long-term central venous catheterization using single lumen, triple lumen, and pulmonary artery catheters. Crit. Care. Med. 1984; 12:634; and 5. Salzman M B, Isenberg H D, Shapiro J F, et al., A prospective study of the catheter hub as the portal of entry for microorganisms causing catheter-related sepsis in neonates. J. Infect. Dis. 1993; 167:487), but their application also includes chest tubes, gas lines, urinary drainages and all post-surgical or interventional drainages and includes needle and needleless access ports, with the later ones having the highest propensity to become colonized and cause infections.

Embodiments of the disclosure include improved designs that build upon known protective devices consisting of protective guards, rib elements and extensions, such as those described in patent DE3816191A1 and related publications (6. Leon C. Alvarez-Lerma F., Ruiz-Santana S., et al, Antiseptic chamber-containing hub reduces central venous catheter-related infection: a prospective, randomized study. Crit. Care. Med. 2003; 31:1318; 7. Walterspiel, J N, Protective Ribs for Male Connectors. Inf. Control and Hospital Epidemiology 1988: 9(8): 342; 8. Walterspiel, J N, Protective Ribs for Connectors, Inf. Control and Hospital Epidemiology 1986; 7(11): 564). Protective guards and extensions on fluid or gas couplings can now, by design, be compressed, bent outwards and/or broken off during the process of closing and securing a coupling. This novel feature is that they can never be physically in the way of closing and securing a connection, regardless of the make, shape or form of the respective counter connector or coupling and its securing provisions.

### DESCRIPTION OF PREFERRED EMBODIMENTS

An embodiment includes a connector having a guard can be made of a resilient, pliable, and easily bendable material, compared to the solid and rigid materials that medical grade couplings are made of. An embodiment may comprise a resilient material having indentations and/or thinner parts that allow the resilient material to bend, and combinations of both resilient and rigid materials. Guards, by virtue of their composition and/or design can be easily bent away from or arolund of a counter connector part that may be in their way during secure closing of a connector.

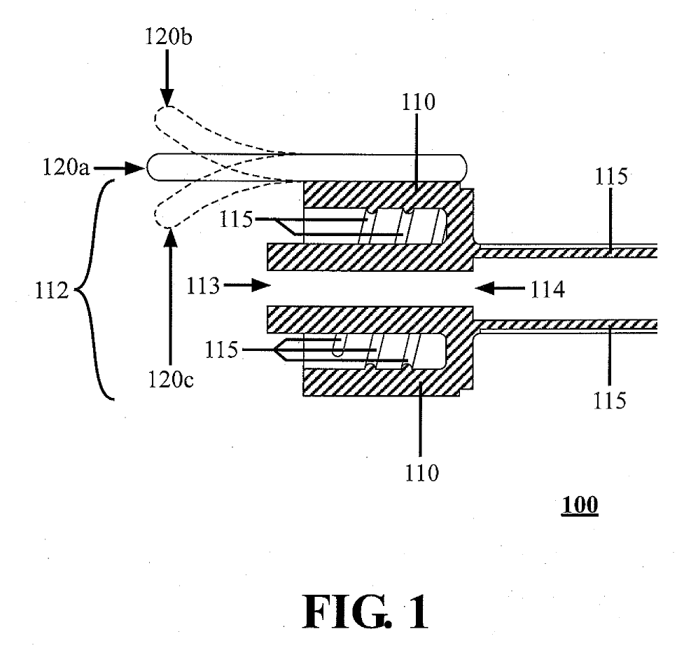

A schematic rendering such a bending embodiment of a guard having a protective rib is depicted in FIG. 1. FIG. 1 depicts a schematic longitudinal section 100 through a male end of a luer connector 112 of this disclosure which includes body element 110 having a series of diagonal threads 115, thereby forming male luer body 112 having a distal end 113 and proximal end 114. Proximal end 114 is connected to tube 105. Flexible rib 120a of a guard is shown in its relaxed position. The rib is depicted bent away from 120b, or towards 120c, or around the axis (not shown) from the male luer connector 112.

An embodiment of a guard having two or more protective ribs can be made of a resilient, pliable, and/or easily bendable material, can be distinguished from the solid materials that medical grade couplings are made of, or where the guard can have indentations and/or a thinner element that can allow it to bend. Guards having protective ribs, by virtue of their composition and/or design can be easily bent away from any counter connector parts that may be in their way during the secure closing of a connector. A guard may comprise one or more ribs mounted on circular rotating ring can be shaped in repeating S forms that can form a receiving opening funnel.

Resilient, flexible materials for guards and ribs can include pliable plastics, such as polyvinyl chloride (PVC) having various amounts and kinds of high and low molecular plasticizers that can include phthalates, polyethylene (PE), and can include various forms and mixtures such as ultra-high-molecular-weight polyethylene, cross-linked polyethylene, medium-density polyethylene, linear low-density polyethylene, low-density polyethylene, very-low-density polyethylene, chlorinated polyethylene and copolymers, polypropylene (PP) in its various forms, including polypropylene glycol (PPG), silicone elastomers in its various forms, including cross-linked polydemethylsiloxane, fluoroplastics, polystyrene, polyethylene terephthalate (PET) and other types of plastics and combinations. Materials may be transparent and/or colored that fulfill the sterility, sterilizability, visibility, and flexibility requirements for said protectors.

The material for the protectors, their clip-on embodiments, and their parts can also include metals, specifically non corrosive alloys. The surfaces can have a hydrophobic coating and color coding.

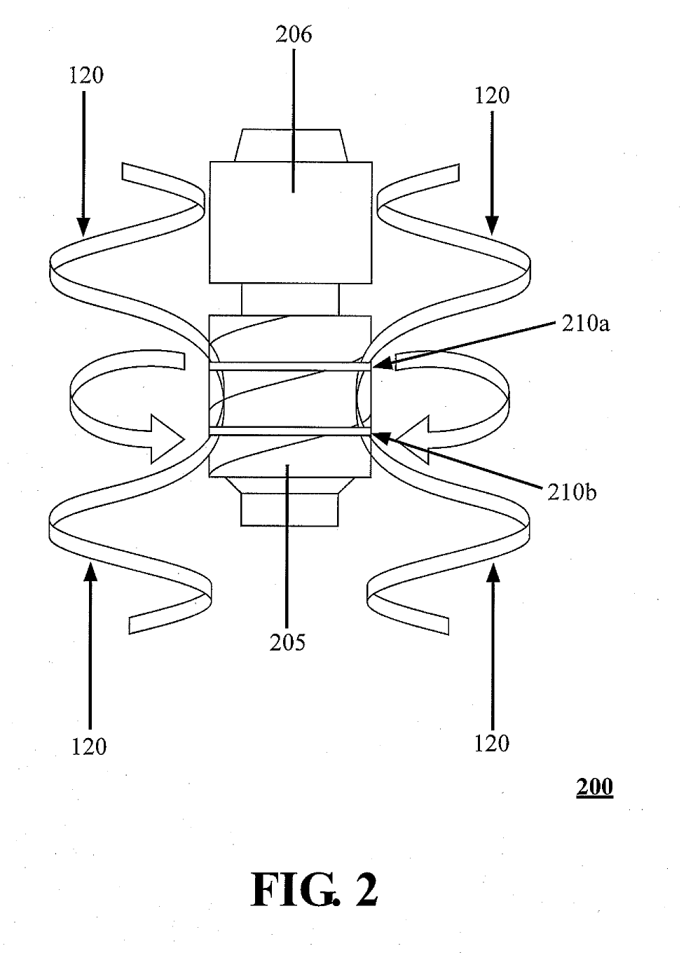

FIG. 2 depicts a schematic drawing of a receiving funnel embodiment of this disclosure comprising a male-female connector having two protective ribs 120a, pointing into their respective distal and proximal directions arranged around a typical luer lock type coupling extender that has a male part 205 below and a female part needleless coupling part 206 above. Shown in FIG. 2 in schematic form are also rotating rings 210a and 210b on of which a rib assembly can rotate (arrows), for the ribs to easily fit into protective assemblies on a counter coupling.

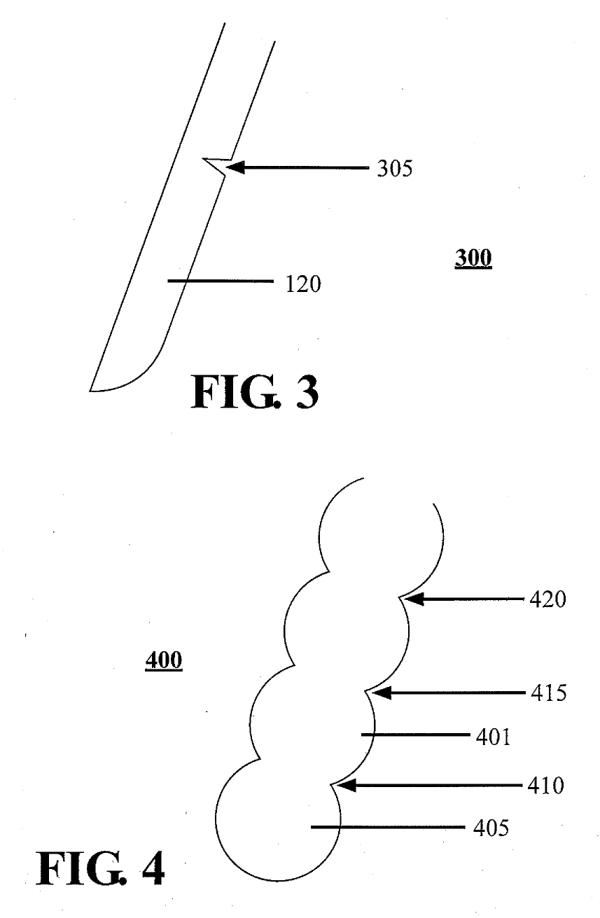

FIG. 3 depicts a schematic drawing 300 of an embodiment of this disclosure showing the distal end of rib 120a. Proximal to the distal end, groove 305 is shown. When rib 120a is deflected away from the Luer connector, it can break at groove 305.

An embodiment of this disclosure has a protective ribs comprising a chain or series of globular or ovoid elements that are connected by thinner areas that can bend and/or break away when coming in contact with a counterpart connector part that may be in their way during the closing of a coupling. Globular or ovoid forms of the elements and combinations thereof can facilitate the slip-over and rotation-over protruding parts of a counter coupling. A schematic drawing of a rib of such an embodiment is depicted in FIG. 4.

FIG. 4 depicts a schematic drawing 400 of an embodiment of this disclosure showing a element of rib 401. Indentations 410, 415, and 420 are show.

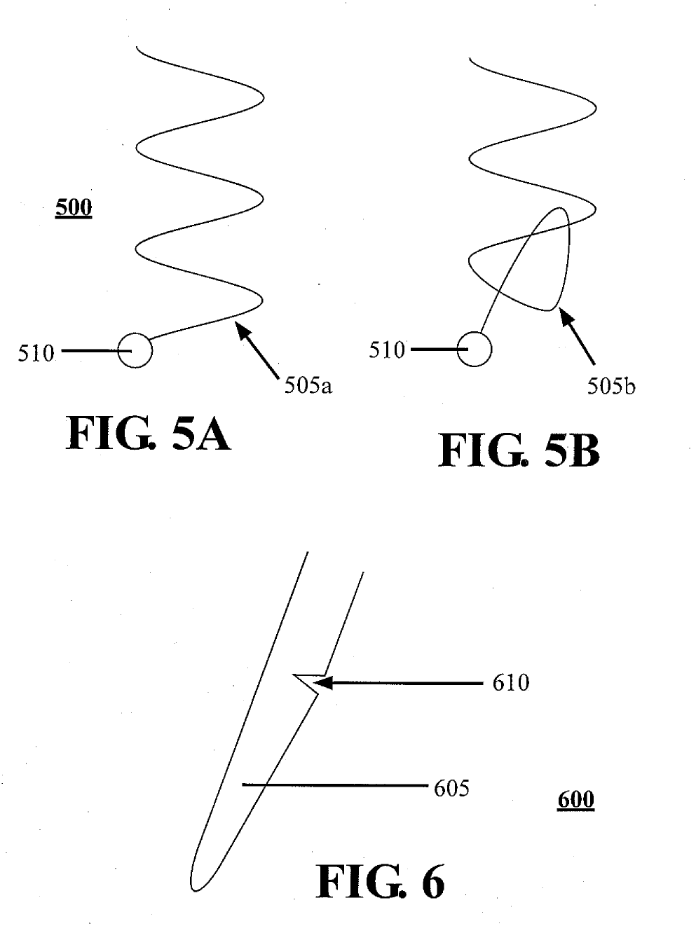

An embodiment of a protective rib cage comprises two or more protecting ribs of undulant shape that can be compressed and/or bend inwards or outwards when the coupling parts are positioned, to or intertwined with each other during the process of closing and securing a luer connector. Two schematic drawings of ribs of such an embodiment are depicted in FIG. 5.

FIG. 5A depicts a schematic drawing 500 of an embodiment of this disclosure having rib 505a in a relaxed state. Element 510 is a looped ending of rib 505a to facilitate sliding over protrusions on a counterpart. Such looped ends of a rib can more easily move out of the way of a protrusion than can a simple pointed end.

FIG. 5B depicts a schematic drawing 500 of an embodiment where rib 505b is deflected to increase its peripheral coverage area, having looped end 505a at its distal end to facilitate sliding over or around protrusions.

An embodiment of a protective rib cage comprises two or more protecting ribs with bending and/or breakpoints whose inner surfaces are slanted at an outward angle, so that said rib, in aggregate, form a funnel with the individual ribs being bent out or even breaking off when the funnel is pried open under the gliding pressure over impeding parts of the counter connecting coupling. An example of a rib in such an embodiment is depicted in FIG. 6.

FIG. 6 depicts a schematic drawing 600 of an embodiment having a slanted rib 605. The distal end of rib 605 is slanted to more easily fit over a complementary connector part. Proximal to the distal end of rib 605, groove 610 is shown. When two complementary elements of a luer connector (i.e., male and female), rib 1201 may break at groove 610.

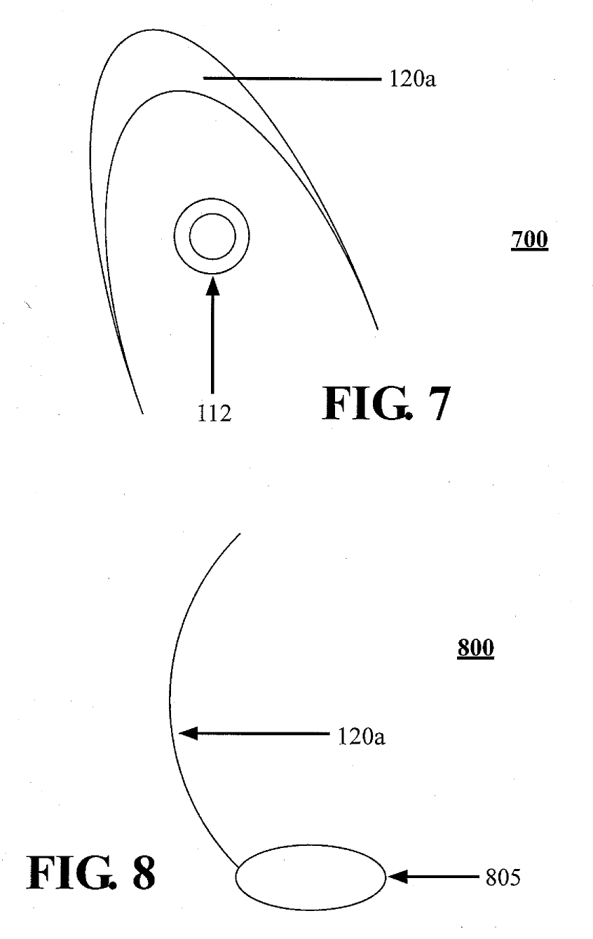

FIG. 7 depicts a cross-sectional view 700 of an embodiment in which a element of a luer connector 112 is surrounded in part by a rib 120a.

FIG. 8 depicts a schematic drawing 800 of an embodiment having rib 120a connected to circular protecting ring 805.

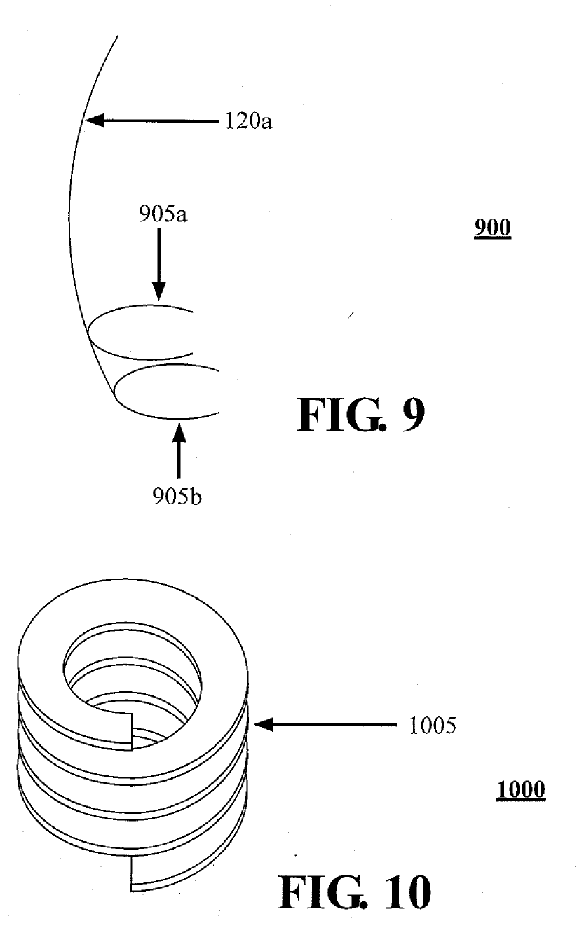

FIG. 9 depicts a schematic drawing 900 of an embodiment of a rib 120a having at a proximal element and two open circular protecting rings 905a and 905b.

FIG. 10 depicts a schematic drawing 1000 of a protective circular guard 1005 of this disclosure that can be compressed out of the way or around of an impeding part during coupling. Such a circular guard can be used on a female connector element so a guard on a male connector element can be inserted inside the circular guard. It can be appreciated that a similar guard may have helical structure.

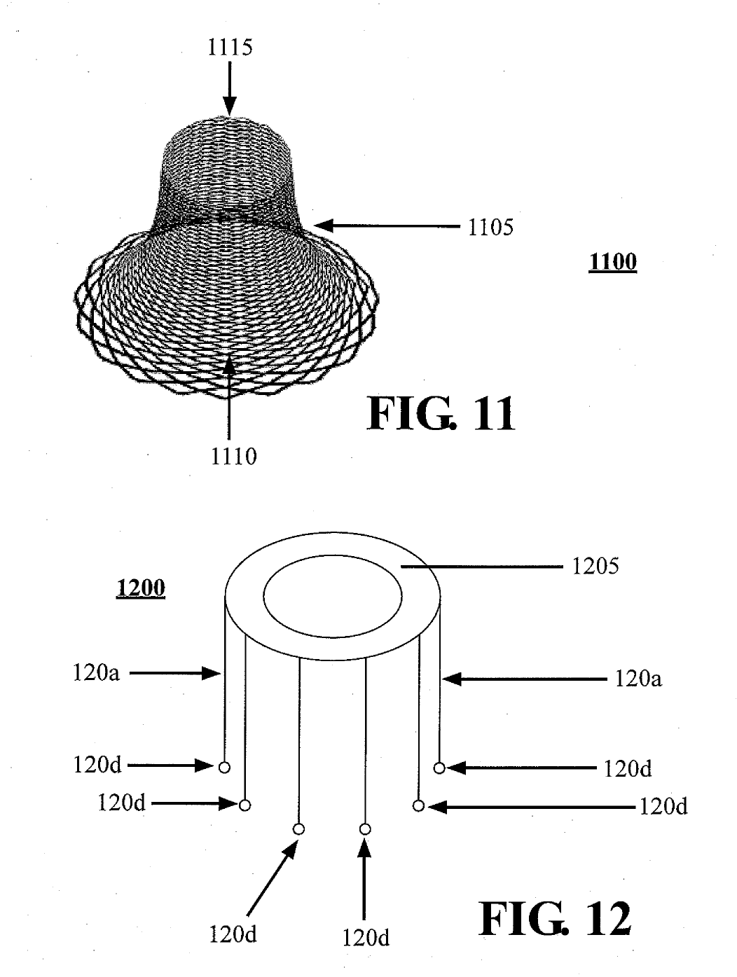

FIG. 11 depicts a schematic drawing 1100 of an embodiment of this disclosure having ribs arranged to form a funnel shaped guard 1105 having a larger diameter opening 1110 and a narrower diameter opening 1115. Narrower diameter opening 1115 can fit over a luer connector part (not shown) to hold it in place relative to the luer connector part, with the larger diameter opening 1110 providing protection for the luer element inside the cage. Such a funnel shaped guard can be placed on a female connector element, and can be sized to permit a male connector element to fit within protective guard 1105.

FIG. 12 shows a schematic drawing 1200 of a protective rib assembly where ribs 120a are arranged on ring 1205 that can be placed in a fitting groove on a connector. End loops 120d at the distal ends of ribs 120a to facilitate sliding of ribs 120a over a protrusion. Ring 1205 can be assembled with a luer connector element to be freely rotatable around the luer connecting element.

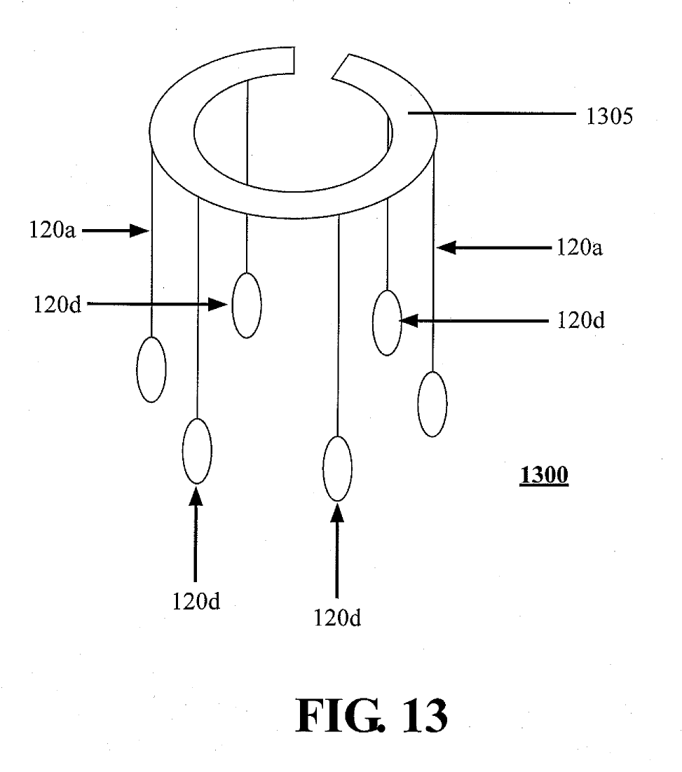

FIG. 13 shows a schematic drawing 1300 of an embodiment, where ribs 120a have loops 120d at their distal ends to facilitate sliding of ribs 120a over a protrusion. Open ring 1305 can be slidably affixed to a luer connector part and axially rotated. This open ring can be clipped into a fitting groove on a connector.

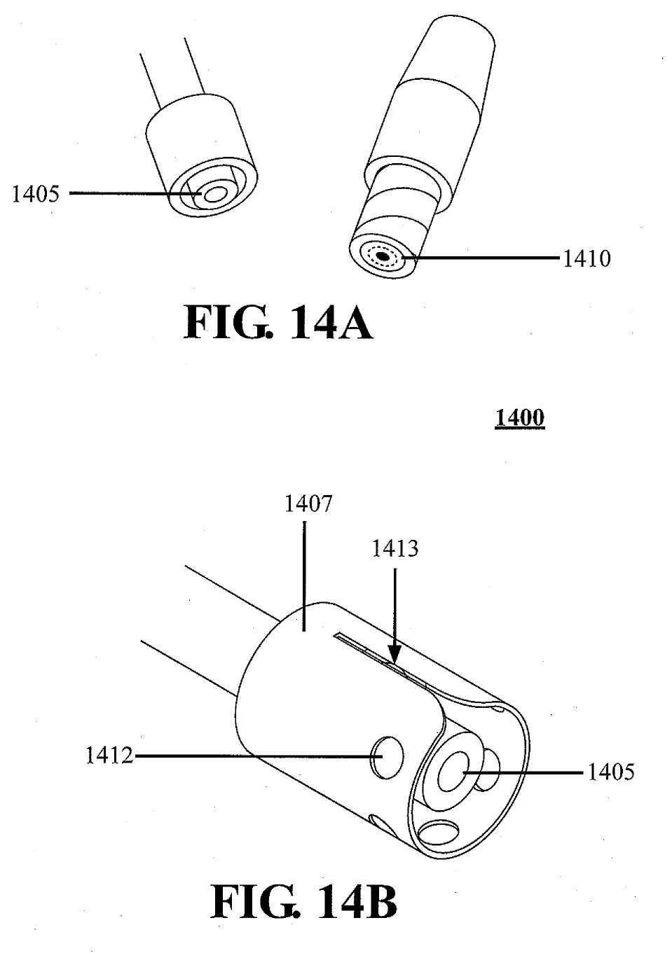

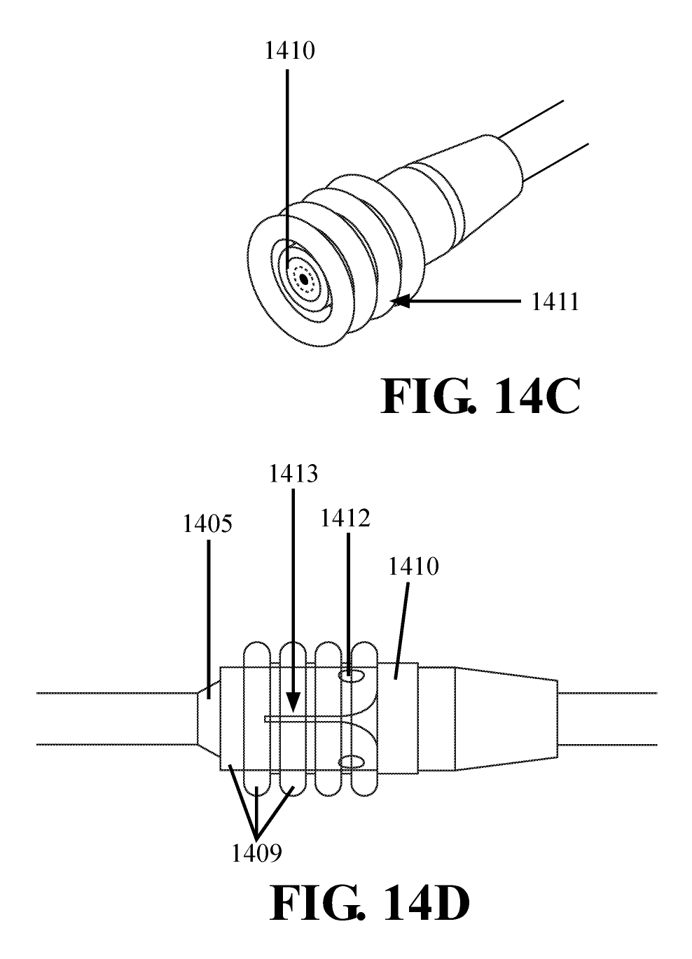

FIG. 14A depicts a drawing 1400 showing male luer connector unprotected element 1405 and female luer needleless connector unprotected element 1410.

FIG. 14B depicts an embodiment of a male luer connector element having a “cage” type protector 1407 (in circle) attached. It can be appreciated that a cage type guard need not have ribs, cut rather, could be manufactured as a single element. Cage type protector 1407 is shown extending beyond the distal end of male luer connector element 1405 thereby making it less likely that the distal end of the male luer connector element 1405 would make contact with a source of contamination. Such openings can increase visibility for proper alignment with a counterpart connector element.

FIG. 14C depicts a drawing of female end 1410 of a needleless connector protected by a compressible protector 1411, where the compressibility of protector 1411 allows for swabbing of exposed end 1410. As with the “cage” type protector illustrated in FIG. 14B, circular protector 1411 extends beyond the distal end of the female luer connector element 1410, thereby making it less likely that the distal end of the female luer connector element 1410 would make contact with a source of contamination. The compressible nature of this protector allows for cleaning a connector part.

FIG. 14D depicts a drawing of an assembled luer connector having male end 1405 and female end 1410 connected together. Male cage protector 1409 enclosing male end 1405 has incision 1412 on its lateral side that allows cage 1407 to have its diameter reduced (to be compressed) when sliding into female protector 1411 or its diameter increased when sliding over a protrusion.

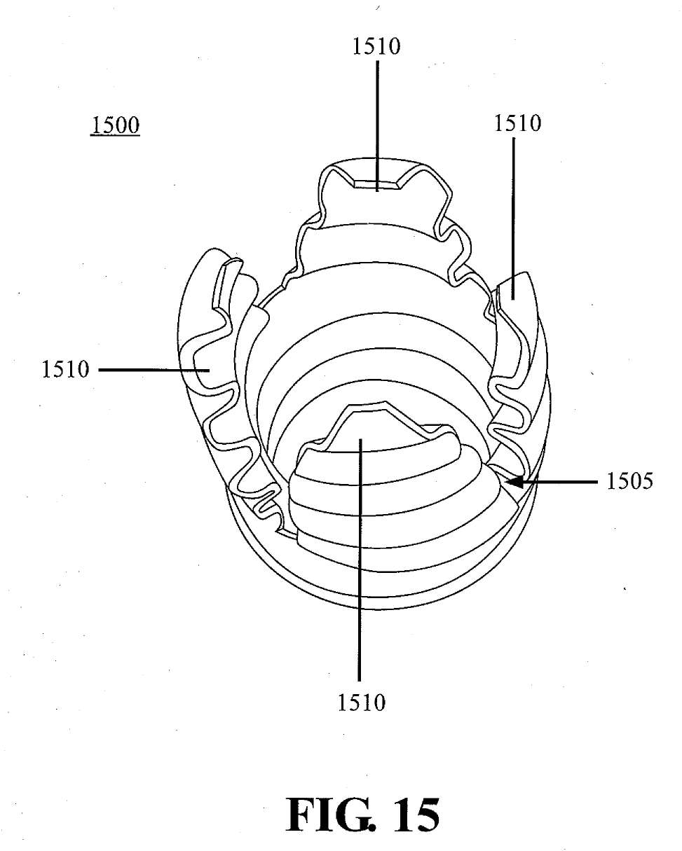

FIG. 15 depicts a drawing 1500 of a guard of this disclosure 1505 having four flap-like elements 1510.

In use, a male element with its cage type protector can have a smaller diameter than the diameter of a female protector, so that a male element can fit inside a female protector. It can be appreciated that a female guard may be sized to have a smaller diameter than a male guard, so a female guard and female element may fit within a male guard.

It can be appreciated that a female protector need not be circular. Rather, it can be made of one or more “stacked” compressible rings, or can be helical.

For avoidance of doubt, the guards and protectors described herein are not limited in use to liquid-liquid connectors. Rather, any fluid-fluid connector can be designed and made based on the disclosure herein for use in any device, including respirators and other gas-gas connectors. Additionally, guards of this disclosure can be used to prevent unwanted contamination in the pharmaceutical and biotechnology industries, where sterility of connectors is desired.

To manufacture connectors with guards, one can obtain commercially available connectors and apply guards of this disclosure to them, thus producing a combined/guard product.

### EQUIVALENTS

A person of ordinary skill in the art can use the disclosures and teachings contained herein to create variations without undue experimentation and with a reasonable likelihood of success. All such equivalents are considered part of this disclosure.

### ADVANTAGES

Embodiments of this disclosure have advantages over existing devices that do not have flexible protective structures. In DE 2843281; reference 7. Walterspiel, J N, Protective Ribs for Male Connectors. Inf. Control and Hospital Epidemiology 1988: 9(8): 342; and 8. Walterspiel, J N, Protective Ribs for Connectors, Inf. Control and Hospital Epidemiology 1986; 7(11): 564, the protectors were rigid. To use rigid protectors, it is necessary to standardize the number of rigid elements in the protector, and they complementary elements (male and female) must be very carefully connected. This can lead to increased difficulty, breakage of the protector elements, and loss of time, and increased likelihood of contamination. Additionally, use of rigid protectors generally require use of a rotating ring, so the male and female components can be screwed together without difficulty.

In contrast, with the use of the flexible, resilient protectors of this disclosure, it is easier to intercalate male and female components together and further reduce the likelihood of contamination of fluid in a luer or other connecting device.

### INDUSTRIAL APPLICABILITY

Improved connectors of this disclosure can find wide use in medical, veterinary, research, biotechnology, chemical industries, and any other industry requiring sterile operations involving connecting fluid-carrying elements to each other.

### REFERENCES

Each of the references cited herein are fully incorporated by references herein as if separately so incorporated.
1. DE 2843281 A1 Oct. 4, 1978 Apr. 10, 1980
2. Linares J., Sitges-Serra A, Garau J., et al., Pathogenesis of catheter sepsis: a prospective study with quantitative and semiquantitative cultures of catheter hub and segments. J. Clin Microbiol 1985; 21:357.
3. Tenney J H Moody M R, Newman K A, et al., Adherent microorganisms on luminal surfaces of long-term intravenous catheters. Importance of Staphylococcus epidermidis in patients with cancer. Arch Intern Med 1986; 146:1949.
4. Miller J J, Venus B, Mathru M., Comparison of the sterility of long-term central venous catheterization using single lumen, triple lumen, and pulmonary artery catheters. Crit. Care. Med. 1984; 12:634.
5. Salzman M B, Isenberg H D, Shapiro J F, et al., A prospective study of the catheter hub as the portal of entry for microorganisms causing catheter-related sepsis in neonates. J. Infect. Dis. 1993; 167:487.
6. Leon C. Alvarez-Lerma F., Ruiz-Santana S., et al, Antiseptic chamber-containing hub reduces central venous catheter-related infection: a prospective, randomized study. Crit. Care. Med. 2003; 31:1318.
7. Walterspiel, J N, Protective Ribs for Male Connectors. Inf. Control and Hospital Epidemiology 1988: 9(8): 342.
8. Walterspiel, J N, Protective Ribs for Connectors, Inf. Control and Hospital Epidemiology 1986; 7(11): 564.

1. A connector comprising:
A mechanically protective resilient guard extending over a male connector part or female connector part used in coupling two portions of a fluid transmitting device, said guard comprising a flexible, pliable material being outwardly bendable, axially compressible, or having easily breakable parts positioned to de not mechanically interfere with the securing of said male and female connector parts to each other.

2. The connector of claim 1, said guard comprising an axially compressible, tubular or spiral sleeve surrounding and longitudinally extending beyond an end of said male or an end of a female connector part.

3. The connector of claim 1, wherein said flexible, pliable material is selected from the group consisting of polyvinyl chloride (PVC), polyethylene (PE), chlorinated polyethylene and copolymers, polypropylene (PP), polypropylene glycol (PPG), a silicone elastomer, cross-linked polydemethylsiloxane, a fluoroplastic, polystyrene, polyethylene terephthalate (PET), metal, and non-corrosive alloy.

4. The connector of claim 3, wherein said flexible, pliable material further comprises a phthalate.

5. The connector of claim 1, said one or more protective guard has elements capable of being bent away from or around a complementary connector part.

6. The connector of claim 1, said one or more protective guard comprising elements having one or more weaker locations that make them breakable in a direction away or towards the center of a coupling.

7. The connector of claim 1, said one or more guards connected to a circular ring adapted to fit over a luer or other connector element.

8. The connector of claim 1, said one or more guards having ribs arranged in outward pointing central slants to form a funnel-shaped protective guard.

9. The connector of claim 1, said one or more protective guards comprising an undulant shape made of a compressible material.

10. The connector of claim 1, said one or more protective guards having a spiral, helical, flat, or tubular shape.

11. The connector of claim 1, said one or more protective guards having comprising interlocking ribs.

12. The connector of claim 1, said one or more protective guards being mounted on one or more rotatable rings.

13. The connector of claim 1, said guard having openings therethrough to permit expansion or compression.

14. The connector of claim 2, said longitudinally axially compressible tubulare sleeve comprises a flexible, pliable material is selected from the group consisting of polyvinyl chloride (PVC), polyethylene (PE), chlorinated polyethylene and copolymers, polypropylene (PP), polypropylene glycol (PPG), a silicone elastomer, cross-linked polydemethylsiloxane, a fluoroplastic, polystyrene, polyethylene terephthalate (PET), metal, and non-corrosive alloy.

15-16. (canceled)

17. A method for connecting ends of a fluid transmitting device to each other, comprising the steps:
a) providing a male connector element of claim 1 of a first tube;
b) providing a female connector element of claim 1 of a second tube;
at least one of said male or said female connector elements having attached thereto one or more protective guards comprising a flexible, pliable material, said one or more guards positioned to extend distally or laterally away from the connector element;
c) moving said male and female connector elements together, where said one or more guards is deflected laterally away from the engageable male and female connector elements; and
d) engaging said male and female connector elements to each other.

18. A method for connecting a male end of a first tube of a fluid transmitting device to a female end of a second tube of a fluid transmitting device, comprising the steps:
a) providing a longitudinally compressible tubular or spiral sleeve of claim 2, said sleeve surrounding and longitudinally extending beyond an end of said male or female connector part;
b) compressing said sleeve to uncover an end of said male or female end; and
c) connecting said male end of said first tube to a female end of said second tube.

19. The method of claim 17, wherein said fluid transmitting device is connected to a gas delivery device or an intravenous line.

20. The method of claim 17, where said fluid transmitting device transmits a biological fluid, culture fluid, or sterile liquid.

21. The method of claim 18, where said fluid transmitting device transmits a gas, a biological fluid, culture fluid, or sterile liquid.
